(19) United States
(12) Patent Application Publication
(10) Pub. No.: US 2012/0094331 A1
(43) Pub. Date: Apr. 19, 2012

(54) METHOD FOR THE PREPARATION OF CARBOHYDRATE CLEAVAGE PRODUCTS FROM A LIGNOCELLULOSIC MATERIAL

(75) Inventors: Karin Fackler, Wein (AT); Kurt Messner, Wien (AT); Chularat Krongtaew, Nonthaburi (TH); Ortwin Ertl, Graz (AT)

(73) Assignee: ANNIKKI GMBH, Graz (AT)

(21) Appl. No.: 13/318,140

(22) PCT Filed: Apr. 30, 2010
(86) PCT No.: PCT/AT10/00137

§ 371 Date: Dec. 20, 2011

(30) Foreign Application Priority Data
Apr. 30, 2009 (AT) A 670/2009
Sep. 23, 2009 (AT) A 1497/2009
Dec. 23, 2009 (AT) A 2030/2009

(51) Int. Cl. C12P 19/00; C12P 7/02

(52) U.S. Cl. 435/72; 435/155

(57) ABSTRACT

A method for the preparation of carbohydrate cleavage products, characterized by the combination of the measures that the lignocellulosic material is treated with an aqueous solution containing hydrogen peroxide, an alcohol, in particular a C₁₋₄ alcohol or a phenol, and a base in order to oxidatively break down lignocellulose and to separate cleavage products from the material, and that the obtained material enriched with cellulose and hemicelluloses is treated with a carbohydrate-cleavage enzyme in order to prepare the carbohydrate cleavage products.

The present inventions relates to a method for the preparation of carbohydrate cleavage products, in particular sugars such as pentoses and hexoses, from a lignocellulosic material. The invention further relates to a method for the production of alcohol from sugars. For the purpose of the present specification and patent claims, the term “sugars” is intended to also include “sugar oligomers”.

In connection with the shortage of crude oil and the discussion on corn as an energy resource, the renewable resource lignocellulose (straw, wood, paper waste, etc.) is gaining importance as a starting material for fuels or chemical products. The conversion of the lignocellulose may be realized by various ways: 1) the “thermochemical platform”, wherein the lignocellulose is initially gasified, and the synthesized gasses are synthesized into the desired products, and 2) the “sugar platform”, wherein the main interest is the use of the sugar bound in the polymers cellulose and hemicellulose, whereas the lignin is still primarily used in an energetic form. The present invention may be assigned to the second way.

In contrast to starch, the sugars of the lignocellulose are present in closely cross-linked, polymeric, crystalline structures of the cellulose and hemicelluloses, additionally surrounded by a lignin coating, this resulting in an extremely dense complex. The most obvious way to prepare sugar from lignocellulose would be the direct use of cellulases and hemicellulases. This, however, in the case of the raw material straw or wood, is exacerbated because of the density of the above mentioned complex. Due to their high molecular weight enzymes are unable to penetrate through the tight pores into the lignocelluloses. This means that it is necessary to carry out a first step for increasing the porosity of the lignocelluloses and thus enabling their further enzymatic saccharification.

This first step is designated as “pre-treatment” (also pulping). It definitely is rather complex, so that, e.g. in the production of “second generation biofuels” up to ⅓ of the production cost must be used for this purpose, which exerts negative influence on the profitability. The methods used aim at either primarily liquidifying the hemicelluloses (i.e., steam explosion, dilute acid pre-treatment), or achieving an increase of the porosity by liquefaction of lignin (i.e., lime, ammonia pre-treatment).

The pulped (decomposed) lignocellulose substrate may be further enzymatically processed for preparing sugar or its oligomers, whereby the type of pre-treatment having substantial influence on the enzymatic activity and the yield. At high reaction temperatures, frequently there are generated toxic degradation products (e.g. furfural), which may inhibit the yeasts in the case that an ethanol fermentation directly follows, see e.g. Chandra et al., Advances in Biochemical Engineering/Biotechnology, 108:67, 2007; Mansfield et al., Biotechnol. Prog. 15:804, 1999.

These methods have the substantial disadvantage that they are highly energy consumpting and are carried out mainly at temperatures slightly below 200° C.

A technological improvement in this field, for example by development of low-temperature methods (this is, at a temperature below 100° C.), would constitute a rather decisive progress for any material use of the raw material lignocellulose. This is the task of the present invention.

From EP 1 025 305 B1 there is known a chemical method for the depolymerisation of lignin (Cu system). It is based on the catalytic effect of complexed copper in connection with hydrogen peroxide or organic hydroperoxides, and it is able to oxidatively cleave lignin at temperatures below 100° C. The complexing agents used therein are pyridine derivatives. By means of lignin models it was possible to confirm that the use of H₂O₂ as an oxidizing agent results in the cleavage of ether bonds of the lignin molecule, by means of which the lignin polymer disintegrates into oligomeric sub-units. By using the Cu system with an excess amount of hydroperoxides it is possible to delignify wood. It appears that the system on the basis of H₂O₂ may be realized technically in a better way, it was tested as a bleaching additive in the peroxide bleaching of Kraft cellulose material and it lead to an improved delignification rate and a higher whiteness.

Furthermore, from “Oxidation of wood and its components in water-organic media”, Chupka et al., Proceedings: Seventh International symposium on wood and pulping chemistry, Vol. 3, 373-382, Beijing P.R. China, 25-28 May 1993, it is known that the efficiency of an alkaline catalysis of the oxidation of wood and lignin is significantly increasing, if an organic solvent, e.g. DMSO, acetone, ethanol is added to the aqueous reaction medium. Furthermore, the authors inform that at pH values above 11, there takes place a sharp rise of the oxidation of the wood and the lignin.

From WO 01/059204 there is known a method for the production of a chemical pulp in which the starting material is subjected to a pre-treatment, wherein the material is treated with a buffer solution and a delignification catalyst (transition metal). The delignification is carried out in the presence of oxygen, hydrogen peroxide or ozone.

In contrast, the method according to the invention for the preparation of carbohydrate cleavage products is characterized in that
lignocellulosic material is treated with an aqueous solution containing hydrogen peroxide, an alcohol, in particular a C₁₋₄ alcohol or a phenol, and a base in order to oxidatively break down lignocelluloses and separate cleavage products from the material, wherein there is obtained material enriched with cellulose and hemicelluloses, and
the obtained material enriched with cellulose and hemicellulose is treated with a carbohydrate-cleaving enzyme in order to prepare carbohydrate cleavage products.

Aliphatic or cyclo-aliphatic, monovalent or polyvalent alcohols or phenols are suitable as an alcohol; e.g. C₁₋₆ alcohols, in particular a C₁₋₄ alcohol, such as methanol, ethanol, propanol and butanol, including the isomers thereof, glycols (ethanediols, propane-, butane-, pentane-, hexanediols), glycerine, propenol, butenol, cyclopentanol, cyclohexanol, benzyl alcohol; or phenols such as phenols, cresols, catechols, naphthols but also amino alcohols such as ethanol amine, methanol amine and hexanol amine. Preferred is a C₁₋₄ alcohol. For the purpose of the present invention, phenols are included in the generic term “alcohol”.

The alcohol solution of the lignin extract furthermore offers advantageous options in the further processing of the lignin, or xylan, respectively, cleavage products.

Hydrogen peroxide is present in the aqueous solution preferably in an amount of 0.1 to 5% by weight, especially preferably in an amount of 0.3 to 2% by weight, for example 0.3 to 1% by weight.

Alcohol is present in an aqueous solution in the method according to the invention, preferably in an amount of 10 to 70% (vol/vol), for example 20 to 50% (vol/vol), preferably 30 to 40% (vol/vol).

In the method according to the invention the lignocellulosic material is present in the aqueous solution preferably in a material density of 3-40% by weight, such as 5-40% by weight, in particular 5-20% % by weight.

Preferably, the lignocellulose is cleaved at a temperature below 100° C., such as below 80° C., for example below 60° C.

The present invention is based, on the one side, on the finding that a lignocellulosic material treated with an aqueous, basic hydrogen peroxide solution, which contains one of the alcohols mentioned above, in particular a C₁₋₄ alcohol or a phenol, may be enzymatically processed with a higher yield into carbohydrate cleavage products, such as sugars, than a material delignified in any other way, in particular without the addition of alcohol.

As carbohydrate cleavage products there are primarily formed sugar, mainly pentoses and hexoses. Preferred sugars include xylose and glucose.

A preferred embodiment of the method according to the invention is characterized in that the material enriched with cellulose and hemicellulose is treated with a xylanase and a cellulase for the preparation of sugar.

As a lignocellulosic material there is preferably used straw, energy crops such as switch grass, elephant grass or abacasisal, bagasse, or untypical lignocelluloses substrates such as bran, for example rice husks, preferably straw, energy crops, bagasse or bran, especially preferably straw or bagasse. Straw has a strongly hydrophobic surface, so that wetting with aqueous solutions is a problem. It has been shown that it is possible by means of using alcohol, to introduce even without pressure the reaction solution into the pores of the substrate and to replace the air present by reaction solution. Furthermore it has been shown that with the selected reaction conditions alcohol accelerates the extraction of the cleavage products from straw and that it contributes to maintaining the lignin cleavage products in solution. Furthermore, it has been shown that, in contrast thereto, alcohol will decrease the solubility of the hemicellulose and the cleavage products thereof and, hence, maintain the hemicelluloses in the substrate. If metal ions are to be introduced with the straw, which in part destroy the hydrogen peroxide, there should be added a complexing agent for the metal ions.

A preferred variant of the methods according to the invention is, that before the treatment of the lignocellulosic material the pH of the aqueous solution is less than 12.0, in particular less than 11.0 and higher than 10.0; furthermore, during the treatment there is not added a base. This is in particular advantageous for the enzymatic processing of the sugars to alcohol, as it has been shown that the pH is decreasing during the treatment, so that there are required only a few chemicals for adjusting the optimal pH for the subsequent enzymatic cleavage of the carbohydrates and for the fermentation of the sugars into alcohol.

By squeezing the liquid phase from the substrate following the pulping process, the substrate concentration is increased so that smaller amounts of enzymes are required for the enzymatic hydrolysis, or in the case of other enzymatic processing steps, respectively.

In the production of alcohol, the enzyme costs are a decisive factor.

Alcohol causes, that the solubility of the hemicelluloses which eventually are released in addition to the lignin and cleavage products thereof in the alkaline range during the reaction, is significantly decreased and these remain bound to the substrate. The advantages for the process are high selectivity of the lignin degradation, in the case of a separation of the extraction solution from the solid, a rather low concentration of hemicellulose and the cleavage products thereof in the extraction solution, because the hemicelluloses remains in the solid portion and, in this way, is available for the enzymatic hydrolysis and sugar preparation.

The alcoholic solution of the lignin extract furthermore offers improved opportunities in the further processing of the lignin and the preparation of lignin products:

By means of the delignification carried out in the pulping process, the porosity of the cell walls of the lignocellulosic material is increased, for example in the case of straw it is increased to such an extent, that nearly the entire xylose becomes accessible to the xylanase and approximately 100% of the xylan may be hydrolyzed and xylose may be obtained. This makes the method according to the present invention in particular suitable for the production of higher-quality products in connection with an enzymatic conversion of the xylose. The enzymatic conversion may be carried out either directly in the mixture of xylose solution and solid, or with the xylose solution separated from the solid.

In a further alcohol production from the remaining solid, following the enzymatic hydrolysis of the xylan and the conversion of the xylose to xylitol according to the invention, the enzyme costs are a decisive cost factor. This result, in part, also from non-specific bonds of enzymes to the lignin, see, i.e., Chandra et al., 2007, ibidem. The partial removal of the lignin reduces this loss of activity and has favourable effects on the costs.

The advantages for a subsequent enzymatic process are, for example, that, because of the high selectivity of the lignin degradation with nearly complete maintenance of the sugar polymers, there will result a rather low concentration of hemicellulose and cleavage products thereof, the hemicelluloses remain in the solid portion and thus remains available for the enzymatic hydrolysis and sugar production as well as the further conversion thereof. This result, according to the invention, in a maximal material use rate and, for example in connection with the use of xylose hydrogenases, to a high profitability of the processes described.

A conversion process of xylose to xylitol may be carried out following the enzymatic release of the xylose directly in the solid-liquid mixture which is obtained according to the present method according to the invention, thus further increasing the profitability of the entire process.

In the case of a conversion to xylitol the residual alcohol from the pulping (decomposition) process, present in the substrate upon squeezing the solid, may be used directly as a substrate for the alcohol dehydrogenase for the regeneration of NAD to NADH. If the process is carried out in such a way, that the residual alcohol from the pulping process which remains in the reaction mixture is (partially) used, the removal of alcohol from the product solution becomes (partly) unnecessary, and the efficiency of the entire process may thus be increased.

In the case of the conversion of the lignin cleavage products, the alcohol acts as radical scavenger and as a solvent for cleavage products from an enzymatic, biomimetic or chemical depolymerisation of the higher-molecular lignin cleavage products to lower-molecular ones.

The small amount of hemicellulose and the cleavage products thereof in the extract and the increased solubility of the lignin increase the flow rates in the case of a separation of the solid from the conversion products, as well as their processing by means of filtration.

The method according to the invention, for example, allows for the separation of the three main components of the straw, this is glucose, xylose and lignin in very contamination-poor material flows and further conversion thereof into higher-quality products, such as xylitol and thus fulfils the requirements of an ideal biorefinery method.

Another advantage of the method according to the invention in comparison with other pulping methods which are mainly carried out in a temperature range between 150° C. and 200° C., is, that it is possible to maintain a reaction temperature below 100° C. The small energy efforts allow for using the lignin obtained in the decomposition process not as an energy source for the decomposition process but rather as a reusable material.

Following the treatment with the aqueous solution containing an alcohol, in particular a C ₁₋₄ alcohol or a phenol and H₂O₂, according to the method of the present invention the solution containing the lignin is separated and the pulped solid is preferably treated with a xylanase, for example for 6-72 hours at 30-90° C. and the liquid phase is separated from the solid, whereby the liquid phase is preferably further converted into secondary products such as, e.g. xylitol.

The solid remaining upon separation of the liquid phase is preferably treated with cellulase, whereby by means of further fermentation of the solid/glucose solution ethanol, butanol or other fermentation products may be obtained; or the remaining solid is subjected to a thermal or thermo-chemical conversion, and the resulting products such as fuel components, fuel additives and/or other chemical products such as phenols, are then separated; or the remaining solid is subjected to a microbial conversion by means of bacteria, yeasts or fungi; or the remaining solid is subjected to a further delignification step in order to obtain a cellulose fibre material.

The remaining solid may be fermented in a biogas plant and further processed into biogas.

One of the secondary products of the xylose that is the most interesting one in an economic aspect is xylitol.

The main sources for the preparation of xylose are cooking liquors originating in the cellulose material industry containing a variety of degradation products, mainly of the lignin and the hemicellulose, so that xylose has to be prepared by means of rather complex separation and purification steps. For example, H. Harms describes in “Willkommen in der natürlichen Welt von Lenzing, weltweit führend in der Cellulosefaser Technologie”, Autumn conference of the Austrian paper industry, Frantschach (15 Nov. 2007) the preparation of xylose from the thick liquor by means of gel filtration, a technically rather complex method that is usually not used for bulk products. The xylose prepared in that way is then catalytically converted into xylitol.

In a further aspect the xylose obtained according to the present invention is converted fermentation-free into xylitol, by conversion with a xylose reductase, e. g. a xylose dehydrogenase, for example from Candida tenuis, wherein there are optionally added a xylose reductase and optionally a co-substrate for regeneration of the co-factor and optionally alcohol dehydrogenase and optionally NAD(P)H to the xylose solution; in particular, wherein the obtained xylose is separated from the lignin cleavage products by filtration.

By way of the following example 1 and the comparative example 1A the influence of the pre-treatment in the presence of alcohol on the yield of reducing sugar upon enzymatic hydrolysis is documented.

### EXAMPLE 1

### Pre-Treatment of Wheat Straw

Wheat straw is crushed to a particle size of about 2 cm. 5 g of crushed wheat straw is suspended in a 500 ml reaction vessel containing a solution consisting of 49.5% water, 50% ethanol and 0.5% hydrogen peroxide. The suspension is heated to 50° C. in a water bath, thermally calibrated, and the pH of the suspension is adjusted with an aqueous NaOH solution to a starting pH of 12. The mixture is continuously magnetically stirred at 200 rpm, 60° C., for 24 hours, then filtered and the solid portion is washed with 11 of distilled water.

For the enzymatic hydrolysis for each parallel tests 100 mg of the pre-treated substrate were adjusted to a pH of 4.8 with 9.8 ml of 50 mM Na-acetate buffer and 200 μL accellerase 1000 suspension (www.genencor.com) were added. Accellerase is an enzyme mixture from cellulases and hemicellulases. Enzymatic hydrolysis was carried out at 50° C. in a shaking water bath. The soluble monomers of hexoses and pentoses released after 48 hours were determined in the form of reducing sugars according to the DNS method (Miller et al., Analytical Chemistry 31 (3):426, 1959) in 1 mL liquid supernatant, based on the amount of the weighed and pre-treated substrate, and expressed in percentage of the maximum theoretical yield.

The theoretical maximum yield of the reducing sugars was separately determined and is 705 mg+/−5% per g of untreated straw.

For each test approach, there were carried out respective 5 parallel tests. The yield of reducing sugars was 99%+/−4%.

### Comparative Example 1A

The above example 1 was repeated, without, however, the addition of alcohol. The yield of reducing sugars was merely 64%+/−3%.

### EXAMPLE 2

### Example 2a

Enzymatic xylitol production from a xylose solution prepared from straw. As a co-substrate there was used isopropanol.

The reaction solution contains 5 mg/mL of xylose.

Xylose reductase (XR) from Candida tenuis reduces xylose to xylitol. This XR requires as a co-enzyme NADH (nicotine amid adenine dinucleotide in reduced form), which is oxidized in the reaction into the co-enzyme NAD⁺. The regeneration of the oxidized co-factor is realized by the parallel activity of an alcohol dehydrogenase (ADH: enzyme-coupled regeneration). Isopropanol is used as a co-substrate. Isopropanol and NAD⁺ are converted by the ADH to NADH and acetone, as shown in reaction scheme 1:

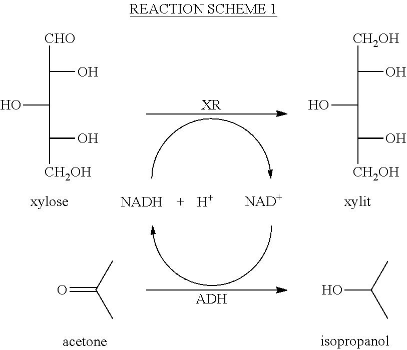

In Table 1 there are set out the reaction ratios in the 5 different test reactions #049, #050, #051, #052, #053 and #054:

TABLE 1

| | Reaction number | | | | |
|---|---|---|---|---|---|
| | #049 | #050 | #052 | #053 | #054 |
| Substrate batch I [μl] | 250 | 250 | 250 | 500 | 500 |
| XR C. tenuis 2 U/mL [μL] | | 50 | 50 | | 50 |
| 20 mM NADH [μL] | | 50 | 50 | | 50 |
| ADH L. kefir 5 U/mL [μL] | | | 50 | | 50 |
| Isopropanol [μL] | | | 50 | | 50 |
| 50 mM Na-phosphate puffer, pH 7.0 | 750 | 650 | 550 | 500 | 300 |
| [μL] | | | | | |

Total volume: 1 mL
Temperature: 26±2° C.
Magnetic stirrer: 200 rpm
Duration: 15 hours

For the deactivation of the enzymes, all samples were heated to 95° C. for 15 minutes and centrifugated as a preparation for the subsequent HPLC analysis.

### Analysis—HPLC:

Column SUGAR SP0810+pre-column SUGAR SP-G
Detector: refractive index detector
Eluent: de-ionized H₂O
Flow: 0.75 mL/min
Sample amount: 10 μL
HPLC quantification precision: ±10%
   Retention time:
Xylose: 13.97 min
Xylitol: 37.73 min
Isopropanol: 16.69 min
Acetone: 16.54 min

### Results:

The substrate concentration of sample #049 was determined by HPLC and was 0.9 mg/mL.

The reaction mixture #050 contains only xylose reductase (0.1 u/ml) and NADH (1 mM). After the reaction of 15 hours, 0.085 mg xylose were spent. The xylitol concentration was below the detection limit.

Reaction #052 is comparable to reaction #050, with the difference, however, that here there is used the regeneration system. There is a total turnover of the xylose used. Concentrations used: XR (0.1 U/mL), NADH (1 mM), ADH (0.25 U/mL) and isopropanol (5%).

The xylose concentration of the sample #053 was determined as 2.121 mg/mL, which corresponds to the expected xylose concentration.

Reaction #054 is comparable to reaction #052, containing, however, a xylose starting concentration (50% substrates in the reaction) increased by the factor 2. The concentration of the produced xylitol was measured as being 0.945 mg xylitol. Concentrations used: XR (0.1 U/mL), NADH (1 mM), ADH (0.25 U/mL) and isopropanol (5%).

In Table 2 the results of the reaction based on the HPLC measurement data are summarized (xylose spent and xylitol obtained; b.d.l. means “below detection limit”):

TABLE 2

| | Reaction number | | | | |
|---|---|---|---|---|---|
| | 049 | 050 | 052 | 053 | 054 |
| Xylose before the reaction | 0.9 | 0.815 | 0.8 | 2.121 | 1.945 |
| [mg/mL] | | | | | |
| Xylose after the reaction | — | 0.815 | b.d.l. | — | 1.013 |
| [mg/mL] | | | | | |
| Xylose spent in the reaction | — | b.d.l. | | — | 0.932 |
| [mg/mL] | | | | | |
| Production of xylitol [mg/mL] | — | b.d.l. | 0.994 | — | 0.945 |
| Xylitol yield relative to the | — | b.d.l. | 100 | — | 47.9 |
| xylose concentration [%] | | | | | |

### Example 2b

Enzymatic xylitol production from a xylose solution prepared from straw. Ethanol is used as a co-substrate.

The volume of the substrate solution was reduced (see example 2) by means of a rotavapor to 50% in order to increase the xylose concentration (˜10 mg/mL xylose).

The regeneration of the oxidized co-factor was realized by the activity of the xylose reductase (XR) used from Candida tenius and the additional activity of a used aldehyde dehydrogenase from Saccharomyces cerevisiae (Sigma-Aldrich: catalogue number A6338; (EC) Number: 1.2.1.5; CAS Number: 9028-88-0). This is a substrate-coupled as well as enzyme-coupled reaction. Ethanol is used as a co-substrate. Ethanol and NAD⁺ are converted in the first step by the activity of the XR to NADH and acetaldehyde. In the second step, acetaldehyde and NAD⁺ are converted by the activity of the aldehyde dehydrogenase (AldDH) to acetate (see Sigma-Aldrich: catalogue number A6338; or “Characterization and Potential Roles of Cytosolic and Mitochondrial Aldehyde Dehydrogenases in Ethanol Metabolism in Saccharomyces cerevisiae”, Wang et al, Molecular Cloning, 1998, Journal of Bacteriology, p. 822-830). In this case, per mol converted co-substrate there would be generated 2 mol reduction equivalents (NADH) (compare reaction scheme 2).

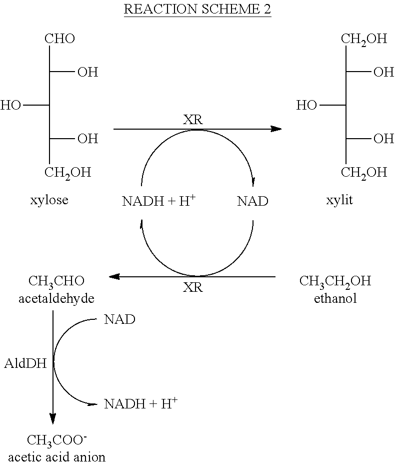

In Table 3 there are set out the reaction ratios of the 4 different test reactions 247, 249, 250 and 253. There were used different ethanol concentrations and AldDH concentrations. The concentrations of the co-factor and the substrate were kept constant.

TABLE 3

| | Reaction number | | | | | | | |
|---|---|---|---|---|---|---|---|---|
| | 247 | | 249 | | 250 | | 253 | |
| Substrate batch III [μL] | 300 | (56 mM) | 300 | (56 mM) | 300 | (56 mM) | 300 | (56 mM) |
| XR C. tenius | 25 | (0.25 U/mL) | 25 | (0.25 U/mL) | 25 | (0.25 U/mL) | 25 | (0.25 U/mL) |
| 5 U/mL [μL] | | | | | | | | |
| 20 mM NAD⁺ [μL] | 10 | (0.4 mM) | 10 | (0.4 mM) | 10 | (0.4 mM) | 10 | (0.4 mM) |
| AldDH S. cervisiae | 25 | (0.25 U/mL | 25 | (0.25 U/mL) | 0 | | 0 | |
| 5 U/mL [μL] | | | | | | | | |
| Ethanol 50% [μL] | 75 | (1286 mM) | 70 | (1200 mM) | 75 | (1286 mM) | 70 | (1200 mM) |

| | | | | |
|---|---|---|---|---|
| 50 mM TrisHCl Puffer | 65 | 70 | 90 | 95 |
| pH 7.0 [μL] | | | | |

Total volume: 0.5 mL
Temperature: 25±2° C.
Thermomixer: 500 rpm
Duration: 112 hours

For deactivation of the enzymes all samples were heated to 70° C. for 15 minutes and centrifugated and filtered as a preparation for the subsequent HPLC analysis (PVDF; 0.2 μm).

### Analysis—HPLC:

Column SUGAR SP0810+pre-column SUGAR SP-G
Column temperature: 90° C.
Detector: refractive index detector
Eluent: deionised H₂O
Flow: 0.90 mL/min
Sample amount: 10 μL
HPLC quantification precision: ±10%

### Results:

The maximum yield (reaction 249) could be achieved with an ethanol concentration of 1.2 mol/L, thereby, in total 1.38 mg/mL of xylitol were produced, which corresponds to a yield of 21.2% of theory of xylitol.

In Table 4 the results of the reactions on the basis of the HPLC measurement data are summarized.

TABLE 4

| | Reaction number | | | |
|---|---|---|---|---|
| | 247 | 249 | 250 | 253 |
| Theoretical total concentration [mg/mL] | 6.288 | 6.407 | 6.268 | 6.150 |
| Xylose after the reaction [mg/mL] | 5.057 | 5.046 | 5.385 | 5.365 |
| Xylose spent in the reaction [mg/mL] | 1.231 | 1.361 | 0.883 | 0.785 |
| Production of xylitol [mg/mL] | 1.248 | 1.379 | 0.894 | 0.796 |

From the results there may be seen that ethanol may be used as a co-substrate. As doubtlessly shown by the comparison of reaction 249 (reaction mixture contains AldDH) and 253 (reaction mixture without AldDH) the addition of the aldehyde dehydrogenase leads to a significant increase of the yield of xylitol. The difference between the turn-over of xylose to xylitol is ˜8%. This result in connection with the above mentioned literature cited leads to the conclusion that AldDH oxidizes the acetaldehyde which is generated in the first partial reduction further to acetic acid (compare reaction scheme 2). This reaction favourable in terms of energy and the increased concentration of NADH associated therewith shifts the balance from the educt in the direction of the product xylitol in the first partial reaction.

1. A method for the production of carbohydrate cleavage products, comprising a combination of measures that
lignocellulosic material is treated with an aqueous solution containing hydrogen peroxide, an alcohol or a phenol, and a base in order to oxidatively break down lignocellulose and to separate cleavage products from the material, wherein there is obtained a material enriched with cellulose and hemicellulose, and
the obtained material enriched with cellulose and hemicellulose is treated with a carbohydrate-cleaving enzyme in order to prepare carbohydrate cleavage products.

2. A method according to claim 1, wherein the cleavage is carried out at a temperature below 100° C.

3. A method according to claim 1, wherein the aqueous solution has a pH before the treatment of the lignocellulosic material that is larger than 10.0 and less than 12.0, in particular less than 11.0.

4. A method according to claim 1 wherein there is not added any base during the treatment.

5. A method according to claim 1, wherein there is used as lignocellulosic material straw, energy crops and/or bran.

6. A method according to claim 1, wherein the lignocellulosic material is present in the aqueous solution in a material density of 5-40% by weight.

7. A method according to claim 1, wherein the material enriched with cellulose and hemicellulose is treated with a xylanase and/or cellulose in order to prepare the sugars.

8. A method according to claim 1, wherein the prepared sugars are fermented to alcohol which is separated and yielded.

9. A method according to claim 1, wherein the solid pulped upon the treatment is converted with a xylanase and that the obtained liquid phase is converted into xylitol, and the remaining solid
is further converted with cellulase to obtain various fermentation products;
or
is subjected to a thermal or thermochemical conversion reaction;
or
is subjected to a microbial conversion by means of bacteria, yeast or fungi;
or
is subjected to a further delignification step for the purpose of the preparation of a cellulose fibre material.

10. A method according to claim 1, wherein the solid pulped upon the treatment is converted with a xylanase and the liquid phase obtained is converted into xylitol using a xylose dehydrogenase, and the remaining solid
is further converted with cellulase to prepare various fermentation products;
or
is subjected to a thermal or thermochemical conversion reaction;
or
is subjected to a microbial material conversion by means of bacteria, yeast or fungi;
or
is subjected to a further delignification step for the purpose of the preparation of a cellulose fibre material.

11. A method according to claim 10, wherein the solid remaining upon the separation of the (fermentation) products is fermented in a biogas plant and further processed into biogas.

12. A method according to claim 9, wherein the solid remaining upon the separation of the fermentation products is fermented in a biogas plant and further processed into biogas.
